(19) United States
(12) Patent Application Publication
(10) Pub. No.: US 2004/0029143 A1
(43) Pub. Date: Feb. 12, 2004

(54) Cationic polyelectrolytes in biomolecule purification and analysis

(76) Inventors: Jeffrey Van Ness, Claremont, CA (US); Lori K. Van Ness, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/377,286

(22) Filed: Feb. 28, 2003

Related U.S. Application Data
(60) Provisional application No. 60/412,442, filed on Sep. 20, 2002.
(60) Provisional application No. 60/360,658, filed on Feb. 28, 2002.

(51) Int. Cl. C12Q 1/68

(52) U.S. Cl. 435/6; 422/58; 436/166

(57) ABSTRACT

Cationic polyelectrolyte comprising multiple quaternary ammonium residues, e.g., poly(diallyldimethylammonium)chloride (PDADMAC) may be used in analytical and separation techniques involving polynucleotides. This polymer and polynucleotides form a strong interaction that may be utilized in separation techniques for polynucleotides, and for stabilizing polynucleotides on a surface.

### TECHNICAL FIELD

The present invention is generally directed to the analysis of biomolecules, including the analysis by, e.g., MALDI mass spectroscopy, and to separation techniques useful in biomolecule analysis, and compositions useful or generated by these techniques.

### BACKGROUND OF THE INVENTION

A comprehensive understanding of biological systems requires knowledge of the chemical composition of cellular systems, and this understanding is becoming increasingly important in elucidating disease mechanisms (Ban, E., Anal. Chem. 70:308A, 1998). Less than 2% of the noninfectious human disease load is monogenic in nature (Strohman, R., Nature Biotechnology 15:194-199, 1997). The remaining 98% is polygenic (involving several genes) in origin or of an epigenetic nature (i.e., nongenetic). An essential step is characterizing the normal range of human polymorphisms (localized changes in a specific DNA sequence in a genome), which provides a necessary benchmark for correlating genetic variance with disease states. Elucidating disease mechanisms requires diagnostic tools for direct DNA sequencing, DNA separation and isolation, and mRNA profiling as well as numerous protein analysis techniques. Analytical methods in this research field must therefore be rapid, accurate, sensitive and robust.

Matrix-Assisted Laser Desorption/Ionization Mass Spectrometry (MALDI-MS) is becoming an ever more popular technique for studying biomolecules (Hillenkamp et al., Anal. Chem., 1991, 63,1193A-1203A). This technique ionizes high molecular weight biopolymers with minimal concomitant fragmentation of the sample material. This is typically accomplished via the incorporation of the sample to be analyzed into a matrix that absorbs radiation from an incident UV or IR laser. This energy is then transferred from the matrix to the sample resulting in desorption of the sample into the gas phase with subsequent ionization and minimal fragmentation. One of the advantages of MALDI-MS over ESI-MS is the simplicity of the spectra obtained: MALDI spectra are generally dominated by singly charged species. Typically, the gaseous ions generated by MALDI techniques are detected and analyzed by determining the time-of-flight (TOF) of these ions. While MALDI-TOF MS is not a high resolution technique, resolution can be improved by making modifications to such systems, e.g., by the use of tandem MS techniques, or by the use of other types of analyzers, such as Fourier transform (FT) and quadrupole ion traps.

MALDI techniques have found application for the rapid and straightforward determination of the molecular weight of certain biomolecules (Feng and Konishi, Anal. Chem., 1992, 64, 2090-2095; Nelson, Dogruel and Williams, Rapid Commun. Mass Spectrom., 1994, 8, 627-631). These techniques have been used to confirm the identity and integrity of certain biomolecules such as peptides, proteins, oligonucleotides, nucleic acids, glycoproteins, oligosaccharides and carbohydrates. Further, these MS techniques have found biochemical applications in the detection and identification of post-translational modifications on proteins. Verification of DNA and RNA sequences that are less than 100 bases in length has also been accomplished using ESI with FTMS to measure the molecular weight of the nucleic acids (Little et al, Proc. Natl. Acad. Sci. USA, 1995, 92, 2318-2322).

In order to conduct an effective MALDI analysis, the nucleic acid to be analyzed must be fairly pure. In most instances, the nucleic acid to be analyzed does not start out pure. For instance, when the nucleic acid is obtained from an assay, the nucleic acid is, at least initially, contaminated with the various components of the assay mixture, which may include various salts and proteins, among other materials. If there is a significant amount of contaminant(s) present with the nucleic acid in the MALDI sample, the contaminant(s) may depress the ion signal through affecting one or more of desorption, ionization and/or detection. The fractionation of an assay mixture to obtain a purified nucleic acid is a time-consuming and expensive process, which typically causes some loss of the nucleic acid to be analyzed, and therefore some loss in signal intensity. In addition, current purification techniques are not readily amenable to automation, where automated MALDI analysis would be very desirable from a cost and efficiency point of view. Accordingly, some improvements in preparing samples for MALDA analysis, particularly where those improvements might be amenable to automation, would be very desirable.

The present invention is based on our discovery that cationic polyelectrolytes, e.g., poly(diallyidimethy)ammonium chloride (PDADMAC), have a sufficiently high affinity to nucleic acids compared to other materials that the cationic polyelectrolytes may be used in rapid and efficient methods for nucleic acid purifications, particularly in connection with MALDI-MS analysis. The following is a summary of literature references that discuss the interaction between biomolecules and an exemplary cationic polyelectrolyte of the present invention, namely PDADMAC.

Wang and Dubin used PDADMAC to coat controlled pore glass for liquid chromatography applications (Wang, Y. and Dubin, P. L., Surfactant Sci. Ser. 80 (Interfacial Phenomena in Chromatography):311-327,1999). The study was primarily implemented to determine a positive or negative effect in protein separations. The coated glass particles were found to enhance protein separations.

Ion-chromatography utilizing PDADMAC as an anion-exchanger with standard reversed-phase chromatography silica beads that have been previously coated with dodecylbenzenesulfonic acid has been reported (Krokhin, O. V., Smolenkov, A. D., Svintsova, N. V., et al. J. Chromatog. A 706:93-98, 1995; Pirogov, A. V., Svintsova, N. V., Kuzina, O. V., et al., Fresenius J. Anal. Chem. 361:288-293, 1998). The anion-exchange PDADMAC layer is bound at the surface of the dodecylbenzenesulfonic acid cation-exchanger by electrostatic interaction of the oppositely charged quaternary ammonium groups (Pirogov et al.).

PDADMAC has been utilized in an independent mode of liquid chromatography called displacement chromatography. Contrary to the more commonly known elution mode of chromatography, in displacement chromatography the components are resolved into consecutive zones of the pure substances rather than into “peaks.” The separation is based on a competition between the sample components for the surface binding places. This competition is enforced during the operation by an advancing displacer front. The displacer is a substance with a higher binding affinity to the stationary phase than the components to be separated. As the displacer front advances, the number of stationary sites available to the binding components is continuously decreased, which engenders competition for the binding sites between the displacer and the components, and also between the components themselves. Under ideal conditions, the more strongly bonded components displace the more weakly bonded ones until all substances are focused into consecutive individual zones of pure substance that leave the column at the speed of the advancing displacer front in the order of the stationary phase affinities.

Displacement chromatography as described in WO 99/47574 utilizes PDADMAC that has been polymerized into a linear water-soluble polymer of a homogeneous structure, narrow distribution of the molar mass, and defined affinity to the stationary phase. This form of PDADMAC polymer is appropriate for inducing the separation of molecules bearing a permanent or temporary positive net-charge or positively charged patches. The preferred PDADMAC polymer average molar mass for displacement was found to be less than 35,000 g/mol and have a narrow molar mass distribution. This size polymer was shown to effectively separate lysozyme and cytochrome c (Schmidt, B., Wandrey, C., Vogt, S., et al., J Chrom. A 865:27-34,1999).

In a non-traditional application of liquid chromatography, PDADMAC was utilized not as a partitioning agent but as a scavenger in a method to separate red blood cells from whole blood (WO 99/36781). Red blood cells in whole blood samples interfere with the determination of the presence or amount of analyte in a blood sample that might otherwise be readily made via an assay system. The PDADMAC is diffusively bound to the stationary phase in a portion of a chromatographic column where it scavenges the red blood cells from a blood sample that has been placed on the column.

The use of buffer additives in capillary electrophoresis to achieve dynamic coating during the separation has also been demonstrated to be a useful method to reduce the basic solute absorption problem. Buffer additives and noncovalent coatings that desorb reversibly may be practical to use in studies in which separating the analyte from the buffer is not important. Chitosan (Yao et al.), fluorosurfactant (Emmer, A., Jansson, M., Roeraade, J., J. Chromatogr. 547:544-550, 1991), PDADMAC (Cohen, N. and Grushka, E., J. Cap. Elec. 1:112-115,1994) as well as other substances have been employed as dynamic reagents added to the buffer solution. Noncovalent additives or coatings have the advantage that manipulation of the coatings is simpler and regeneration of the capillaries is more straightforward than the covalently modified capillaries.

Capillary electrophoresis with a water-soluble ion-exchange polymer, such as PDADMAC, in the background electrolyte has been disclosed as being efficient for the separation of organic and inorganic anions because of the ion-exchange selectivity, differences in electrophoretic mobility, and the ability of the ion-exchange polymer to physisorb to the silica surface. The differential ion-pair formation occurs both in the mobile phase as well as with the ion-exchange polymer coating the silica capillary column (Li, J., Ding, W., and Fritz, J. S., J. Chromatog. A 879:245-257, 2000). Addition of organic solvent could also modify the sample migration and the separation (Li et al).

The applicability of capillaries that are either chemically bonded or physically coated with PDADMAC has been demonstrated with varying results. In some instances, PDADMAC resulted in successful separations. In one example where PDADMAC was both covalently bonded and physically absorbed to the capillary surface, the EOF remained strong enough within the acidic pH range to permit rapid separations of basic proteins and beta-blockers with good reproducibility. (Liu et al.). Although covalent bonding of capillary coatings would seem preferable in terms of durability, similar robustness was observed for both types of coatings in this particular case.

Other studies have had the capillary column coated only. Wang and Dubin found that PDADMAC of sufficiently high molecular mass could be used to give a stable polymeric coating to fused-silica capillaries (Wang, Y. and Dubin, P. L., Anal. Chem. 71:3463, 1999). The positive surface reproducibly reversed the direction of electroosmotic flow. Liu et al. found that capillaries with a PDADMAC polymer coating improved the capillary electrophoresis separation of basic proteins and beta-adrenergic blocking drugs.

Cordova et al. found that PDADMAC did not coat the capillary due to the restricted conformations of its backbone and consequently diminished Coulomb interactions between the polymer and the walls of the capillary (Córdova, E., Gao, J., and Whitesides, G. M., Anal. Chem. 69:1370-1379,1997).

PDADMAC in solution can selectively bind β-lactoglobulin in the presence of bovine serum albumin, even though these two proteins have similar isoelectric points. Wang and Dubin demonstrated that this protein binding selectivity is maintained after PDADMAC is immobilized on a silica capillary wall by physisorption (Wang, Y. and Dubin, P. L., Surfactant Sci. Ser. 80(Interfacial Phenomena in Chromatography):311-327, 1999). In addition, by properly controlling polyelectrolyte adsorption conditions it was found to be possible to change the structure of the immobilized PDADMAC and thus subsequently change the amount of protein binding.

In a slight variation to the above applications, PDADMAC has also been utilized as a copolymer with acrylamide in an electrolyte solution (U.S. Pat. No. 5,264,101). Both polymers remain non-cross-linked. This combination of polymers serves as both a molecular sieving matrix to biomolecules as well as a non-covalent wall coating.

European patent 0209251 describes the use of PDADMAC coupled to a hydroxy-substituted gel for affinity chromatography of antibodies and other proteins. The use of quaternary nitrogen containing polymers was preferred over monoclonal antibodies due to the cost and difficulty in producing the antibodies. Polymeric quaternary nitrogen compounds contain several positively charged sites to interact with the protein of interest. In addition, the polymeric nature gives some control over steric access and thus offers flexibility in applications. The gel polymerization works on the assumption that one or more of the nitrogen atoms of the polymer may still be reactive as they are not all necessarily quaternized. If one nitrogen is not quaternary, the polymer may be coupled to a hydroxy-substituted gel such as agarose, dextran, cellulose, and the like through a bis-epoxy compound. This process can be utilized in gels of all pore sizes.

Kuron et al. utilized PDADMAC for in vitro bile acid sequestering of cholesterol in normocholesterolemic dogs to make the dogs hypocholesterolemic (Kuron, G. W., Grier, N., and Huff, J. W., Atherosclerosis 37:353-60,1980). The complexation of the protein ferrihemoglobin and PDADMAC has been studied using quasi-elastic light scattering, electrophoretic light scattering, circular dichroism spectroscopy and azide binding titrations (Xia, J., Dubin, P. L., Kokufuta, E., et al., Biopolymers 50:153-61,1999). In the presence of excess protein, many ferrihemoglobin molecules bind to the PDADMAC causing a 2.5 fold increase to the PDADMAC diameter and consequently decreasing the mobility of the polymer. In another protein assay, U.S. Pat. No. 5,459,078 utilizes PDADMAC as a capture reagent in a digoxin assay.

The present invention is directed to improvements in analytical techniques that overcome some of the disadvantages found in current methods. These and related aspects of the present invention are describsed in further detail below.

### SUMMARY OF THE INVENTION

The present invention utilizes cationic polyelectrolytes (CPs), preferably polymeric permanent polycations such as poly(diallyldimethylammonium chloride) (PDADMAC) as an active component in the purification of nucleic acid molecules.

In one aspect the present invention provides a plate comprising a surface, the surface being at least partially coated with a coating comprising cationic polyelectrolyte (CP), where, in a preferred embodiment, the CP comprises multiple quaternary ammonium-containing residues, e.g., diallyidialkylammonium residues, where preferably at least 5% of the monomer residues include quaternary ammonium groups. The plate may optionally have wells, e.g., 10 to 1,000 wells, and these wells may optionally be arranged as in standard 96- and 384-well plates. The coating may be in contact with polynucleotide, e.g., the coating may support an array of polynucleotides.

In another aspect, the present invention provides a dipstick comprising a polymeric coating, the polymeric coating comprising cationic polyelectrolytes (CPs), preferably, polymeric permanent polycations such as poly(diallyldimethylammonium chloride) (PDADMAC), or other polymer that has multiple diallyidialkylammonium residues.

In another aspect, the present invention provides a filter comprising a porous matrix, the matrix coated with a cationic polyelectrolytes (CPs), preferably, polymeric permanent polycations such as poly(diallyldimethylammonium chloride) (PDADMAC), or other polymer comprising multiple quaternary ammonium-containing residues, e.g., diallyidialkylammonium residues, where preferably at least 5% of the monomer residues include quaternary ammonium groups.

In another aspect, the present invention provides a method of performing an assay, comprising: (a) providing a plate having wells with distinct indented surfaces, the surfaces coated with a cationic polyelectrolyte, the cationic polyelectrolyte comprising multiple quaternary ammonium-containing residues, e.g., diallyidialkylammonium residues, where preferably at least 5% of the monomer residues include quaternary ammonium groups; (b) performing a nucleotide extension assay in a well of (a), the extension assay optionally incorporating a tagged nucleotide into an extension product where the tag is optionally a fluorescent tag; (c) detecting the presence or absence of tagged extension product by fluorescent spectroscopy.

In another aspect the invention provides a method of performing an assay, comprising: (a) combining an analyte with a cationic polyelectrolyte to provide a mixture; (b) placing the mixture on a substrate to provide a sample; and (c) analyzing the sample by MALDI-MS. As in the other aspects of the invention, the cationic polyelectrolyte may, in one embodiment, comprise quaternary ammonium groups, e.g., the cationic polyelectrolyte may be PDADMAC. MALDI-MS typically requires the presence of a matrix in the sample. In one aspect, the matrix is part of the mixture, while in another aspect the matrix is part of the sample.

In another aspect, the present invention provides a method of isolating nucleic acids, comprising, in sequence: (a) providing a solution comprising polynucleotides; (b) inserting a substrate into the solution, the substrate at least partially coated with a cationic polyelectrolyte, the cationic polyelectrolyte comprising multiple quaternary ammonium-containing residues, e.g., diallyldialkylammonium residues, where preferably at least 5% of the monomer residues include quaternary ammonium groups; (c) allowing the nucleic acids to adhere to the polymer; and (d) removing the substrate and at least some of the nucleic acids from the solution.

In another aspect, the present invention provides a method of purifying nucleic acid, comprising: (a) providing a mixture comprising nucleic acid and non-nucleic acid; (b) providing a solid support comprising a coating comprising cationic polyelectrolyte; (c) contacting the mixture with the cationic polyelectrolyte under conditions where nucleic acid adheres to the cationic polyelectrolyte, to provide adhered nucleic acid; (d) separating adhered nucleic acid from at least some of the non-nucleic acid; and (e) characterizing the adhered nucleic acid.

In another aspect, the present invention provides a method for environmental analysis comprising: (a) contacting an environment with a solid support comprising a surface, the surface being at least partially coated with a cationic polyelectrolyte, the cationic polyelectrolyte being contacted with the environment for a time sufficient for the catonic polyelectrolyte to absorb one or more organic compounds from the environment so as to provide a contaminated surface; and (b) analyzing the contaminated surface by MALDI-MS to provide an identification of the one or more organic compounds present in the environment.

In another aspect, the present invention provides a method for detecting the presence or absence of a biomolecule in an environment, comprising: (a) exposing cationic polyelectrolyte to an environment, where the environment may or may not contain an organic biomolecule; (b) allowing the cationic polyelectrolyte to interact with the environment so as to absorb a biomolecule from the environment in the event a biomolecule is present in the environment, thereby providing a potentially contaminated cationic polyelectrolyte; and (c) analyzing the potentially contaminated cationic polyelectrolyte by MALDI-MS to determine whether an organic biomolecule was present in the environment.

In the method for environmental analysis and the method for detecting the presence or absence of a biomolecule in an environment, in various optional embodiments: the organic compound/biomolecule is selected from amino acid, saccharide and nucleotide; the organic compound/biomolecule contains a plurality of residues selected from amino acid, saccharide and nucleotide residues; the organic compound/biomolecule is selected from polypeptide, polysaccharide and polynucleotide; the organic compound/biomolecule is indicative of a health hazard in the environment; the organic compound/biomolecule is indicative of a pathogen. Also, independently, in various optional embodiments of the method for environmental analysis and the method for detecting the presence or absence of a biomolecule in an environment: the cationic polyelectrolyte is part of a composite structure; the cationic polyelectrolyte is part of a composite structure that comprises a solid support and a coating of cationic polyelectrolyte on the solid support; the cationic polyelectrolyte comprises quaternary ammonium groups; the cationic polyelectrolyte is PDADMAC. In addition, independently, in various optional embodiments of the method for environmental analysis and the method for detecting the presence or absence of a biomolecule in an environment: the environment is an air supply; or the environment is a water supply.

In other aspects, the present invention provides kits for analyzing organic molecules and biomolecules in an environment. For example, the invention provides a kit for detecting the presence of an organic biomolecule in an air supply, comprising: (a) a solid support comprising a cationic polyelectrolyte; and (b) a means for securing the solid support in the air supply. As another examples, the invention provides a kit for detecting the presence of an organic biomolecule in a water supply, comprising: (a) a solid support comprising a cationic polyelectrolyte; and (b) a means for securing the solid support in the water supply. The these kits, in one aspect, the cationic polyelectrolyte comprises quaternary ammonium groups, e.g., the cationic polyelectrolyte is PDADMAC.

These and related aspects of the present invention are set forth in detail below.

### BRIEF DESCRIPTION OF THE DRAWINGS

FIG. 1 is a MALDI-TOF spectrum of 10 fM Glu-fibrinopeptide. A single large peak is observed at a m/z of 3644.618. A minor sodium adduct of the peptide is observed at 3666.092 m/z.

FIG. 2 is a schematic illustration of an amplification method that may be characterized by methods of the present invention.

FIG. 3 is a MALDI-TOF spectrum of a genotyping reaction. The peaks at 3694 m/z and 3761 m/z represent the forward and reverse fragments generated in the amplification process that incorporate the SNP. The SNP generated was the G allele. The peaks at 4007 m/z, 4354 m/z, 7015 m/z, and 7838 m/z are polynucleotides generated in the amplification process that do not incorporate the SNP information.

### DETAILED DESCRIPTION OF THE INVENTION

In one aspect, the present invention provides a composition comprising polynucleotide and a polymeric cationic polyelectrolyte. The inventive compositions have unique properties that render them particularly useful in a wide variety of methods. Notably, these methods take advantage of the relatively high binding energy observed when polynucleotide is combined with the polymer.

As used herein, “polynucleotide” refers to any nucleic acid-containing molecule, e.g., deoxyribonucleic acid (DNA) and ribonucleic acid (RNA), and includes oligonucleotides, fragments generated by the polymerase chain reaction (PCR), and fragments generated by any of ligation, scission, endonuclease action, and exonuclease action. Polynucleotides can be composed of two or more monomers that are naturally-occurring nucleotides (such as deoxyribonucleotides and ribonucleotides), or analogs of naturally-occurring nucleotides (e.g., α-enantiomeric forms of naturally-occurring nucleotides or base analogs), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleotides can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “polynucleotide” includes oligonucleotides (i.e., polynucleotides having less than 100 nucleotides) incorporating one or more specificity spacers (as defined herein) where abasic residue and base analog residues are exemplary specificity spacers. The term “polynucleotide” also includes so-called “peptide nucleic acids,” which comprise naturally-occurring or modified nucleic acid bases attached to a polyamide backbone. Polynucleotides can be either single stranded or double stranded. In a preferred aspect of the invention, the polynucleotide is single-stranded.

When the cationic polyelectrolyte is used to separate polynucleotide from a cell lysate or other natural system, the polynucleotides may have many thousands of nucleotides, even 10⁶ nucleotides. However, when the polymer is used to separate polynucleotides that are generated in an assay, the polynucleotides typically have less than 1,000 nucleotides, and may have less than 100 nucleotides.

In one aspect of the present invention, the polynucleotides are DNA. In another aspect, the polynucleotides are RNA. In another aspect, the polynucleotides are a mixture of DNA and RNA. In another aspect, the polynucleotides are PCR reaction products.

The present invention utilizes a polymeric cationic polyelectrolye. As used herein, the term “cationic polyelectrolyte” (CP) denotes a high molecular weight polymeric material having, as part of its chemical structure, a plurality of positively charged chemical groups. In a preferred embodiment, the CPs have a plurality of ammonium groups that provide the positive charges. Such ammonium groups are either the protonated form of a primary, secondary or tertiary amine, or are quaternary ammonium groups. The CP useful in the present invention preferably has quaternary ammonium groups because such groups retain their cationic character at any pH, in contrast to protonated primary, secondary or tertiary amines that are cationic only under low pH conditions. The molecular weight of the CP (either number or weight average molecular weight) is preferably greater than 10,000 Daltons. In various embodiments, the CP has a molecular weight in excess of 100,000 Daltons, or in excess of 200,000 Daltons, or in excess of 300,000 Daltons.

CPs are typically prepared by either polymerizing amine-containing polymerizable monomers, or by converting an existing high molecular weight material into an amine-containing material. Two well-known CPs that are made by polymerizing an amine-containing monomer are polyethylenimine (PEI) and polydiallyldimethylammonium chloride (PDADMAC), which are the polymerization product of ethylenimine and diallyldimethylammonium chloride, respectively. Also suitable for use in the present invention is the polymerization product of diallyldialkylammonium anions other than diallyldimethylammonium chloride, where other suitable anions include bromide and acetate, and where other suitable alkyl groups include ethyl, n-propyl, i-propyl, n-butyl, i-butyl, t-butyl, etc. 2-Vinylpyridine (2-VP) and 4-vinylpyridine (4-VP) are two monomers that have been polymerized to form a CP, i.e., poly(2-vinylpyridine) and poly(4-vinylpyridine) respectively, although these monomers could also be used in a copolymerizaton process to produce a CP. Allylamine may be polymerized to form poly(allylamine hydrochloride) and vinylamine may be polymerized to form polyvinylamine. Related suitable CPs are polyamidoamines modified by grafting onto ethyleneimine, polyamidoamines, polyetheramines, polyvinylamines, modified polyvinylamines, polyalkylamines, polydimethylaminostyrene, polyvinylimidoazoles, and polydiallydialkyl ammonium halides, in particular polydiallyld imethylammonium chloride.

Many CPs made by monomer polymerization are homopolymers, where PEI and PDADMAC are two notable examples. However, CPs may also be made by copolymerization of two or more monomers. In some instances, only one of the co-monomers has, or will be derivatized to have, an amine group, and the other co-monomer is effectively a diluent for the charge density of the CP. Alternatively, if the amine-containing monomer is not very reactive with itself, a co-monomer may be added to the reaction mixture in order that the amine-containing monomer has something to readily react with. In other instances, the two co-monomers react together so as to form the cationic groups of the polymer, where the reaction product of dimethylamine and epichlorohydrin, commonly known as “polyamine”, is an example of this class. Poly (N,N-dimethylammonio ethylene iodide) (PDMAE) is another example of a CP that is commonly made from two different co-monomers. Also within this category are CPs known as polyamidoamine-epichlorohydrin (PAAE), which are formed by the reaction between adipic acid and diethylene triamine, and subsequent derivatization of the resulting copolymer with epichlorohydrin. Structurally similar is the CP known as polybrene, having the chemical name of (1,5-dimethyl-1,5-diazaundecamethylene polymethobromide, hexadimethrine bromide).

Another group of CPs that are made by monomer polymerization are the cationic polyacrylamides (CPAMs). These are prepared by the polymerization of acrylamides having amine substitution. Related to the CPAMs are polyacrylates and polymethyacrylates (collectively, poly(meth)acrylates, CPATs)) that have a plurality of amine groups. Such poly(meth)acrylates are prepared either by polymerization of a (meth)acrylic ester having an amine group, or by converting a poly(meth)acrylate to an amine-containing polymer. Poly[2-(propionyloxy)ethyl]trimethyl-ammonium chloride (PCMA) and poly[3-(2-methylpropionamido)propyl]trimethyl-ammonium chloride (MAPTAC) are two examples of CPATs and CPAMs, respectively.

Another group of cationic polyelectrolytes, this time prepared by adding amine groups to a high molecular weight material, is the cationic starches (CSs). Starch is a polysaccharide having a plurality of hydroxyl groups. Those hydroxyl groups are reactive with many different functional groups, e.g., epoxide groups, and accordingly may be converted into amine-containing groups. One commercially available cationic starch is formed by reaction of epoxypropyltrimethylammonium chloride and starch. CSs prepared from trialkylammonium-containing epoxides, e.g., dimethylethyl, methyidiethyl, triethyl, etc., may also be used in the present invention.

The CP may be amino-acid based, i.e., may contain a plurality of peptide bonds. Such materials include, without limitation, histones, poly-L-arginine, poly-L-histidine, poly-L-lysine, copolymers of one or more of arginine, histine and lysine with other amino acids, gelatin, and protamine.

Commonly recognized properties of cationic polyelectrolyes are morphology (e.g., linear, branched), molecular weight, charge density, and polydispersity. CPAM and cationic amylose are high molecular weight, linear molecules with a low charge density. PDADMAC may be prepared in either high or low molecular weight forms, but in either case has a high charge density. PEI may also be prepared with a range of molecular weights, but typically is a branched (globular) molecule with a high charge density. Cationic amylopectine is also a branched molecule, but it has a low charge density.

A preferred CP that may be used in any of the compositions and methods disclosed herein has quaternary ammonium groups. A preferred CP having quaternary ammonium groups is poly(diallyldialkyl)ammonium, and particularly preferred is the salt of poly(diallyldimethyl)ammonium, where the chloride salt is commonly known as PDADMAC. Without being bound to this theory, the inventor speculates that the high charge density of PDADMAC contributes to the superior performance property of this CP in the methods and compositions of the present invention. PDADMAC macromolecules tend to be quite stiff and may be expected to have a more extended conformation in solution and on a substrate, where this feature of PDADMAC may also contribute to its superior performance. The molecular weight of PDADMAC that may be used in the present invention is typically in the range of hundreds of thousands of grams per mole up to a million grams per mole. PDADMAC is commercially available in various molecular weight ranges, and is typically provided as a liquid concentrate having a solids level in the range of 10 to 50%.

The CP may optionally be crosslinked. To afford crosslinking to the CP, a crosslinking agent may be added to the monomer reactants used to form the CP. Typically, the crosslinking agent should have two or more reactive groups per molecule, where those reactive groups should be reactive with one or more of the monomers used in the synthesis of the CP. Exemplary crosslinking agents that are suitable for use when the monomers are reactive due to olefin groups include, without limitation: divinylbenzene; N,N-methylenebisacrylamide; triallyl 1,3,5-benzenetricarboxylate (a.k.a. triallyl trimesate, Chemical Abstracts Registry Number (RN) 17832-16-5); poly(ethylene glycol) methacrylate (RN 25736-86-1); and (+)-N,N′-Diallyltartardiamide (a.k.a. (+)-N,N′-diallyltartramide, RN 58477-85-3).

The cationic polyelectrolyte is selected to have high affinity for the polynucleotide, and therefore is useful in purification and/or separation of polynucleotide molecules.

In various aspects, at least 10%, at least 20%, at least 30%, at least 40%, at least 50%, at least 60%, at least 70%, at least 80%, at least 90%, at least 95% or 100% of the monomer residues present in a polymeric cationic polyelectrolyte comprise quaternary ammonium groups, preferably quaternary ammonium groups derived from the diallyldialkylammonium monomer. In one aspect, the cationic polyelectrolyte is a homopolymer. In general, as the cationic polyelectrolyte contains more quaternary ammonium groups, the strength of the interaction between the cationic polyelectrolyte and the polynucleotide increases. A strong interaction between the cationic polyelectrolyte and polynucleotide is generally preferred in the methods to which the composition is preferably utilized. The use of quaternary ammonium groups, rather than other positively charged nitrogen species, e.g., protonated tertiary amines, is generally advantageous in that quaternary ammonium groups have charges that are relatively insensitive to changes in pH and salt.

While at least 5% of the monomer residues include quaternary ammonium groups, the polymeric cationic polyelectrolyte may contain residues that do not comprise quaternary ammonium groups. Thus, the cationic polyelectrolyte may be a homopolymer or a copolymer, where the copolymer may be a block or random copolymer. When the polymer is a copolymer, the monomer residues that do not include quaternary ammonium groups may, instead, have any other type of functionality. The functionality may simply be a hydrogen. For instance, the residue containing the quaternary ammonium groups may be adjacent to a residue formed from ethylene. Alternatively, the residue containing the quaternary ammonium group may be adjacent to a residue from other olefinic reactants, including propylene, butylenes and other alpha-olefins; dienes including butadiene and styrene, aromatic residues such as styrene, and acrylates, including methacrylates and esters of acrylates and methacrylates.

The cationic polyelectrolyte may, or may not, be crosslinked. Crosslinking may be advantageous in those instances where mechanical strength is important. That is, in general, a crosslinked polymer has a greater mechanical strength than a non-crosslinked polymer. Thus, in those instances where enhanced mechanical strength is desired, a crosslinked polymer may be employed. Crosslinking may also enhance the amount of surface area that is covered by the polymer, and so to enhance coverage, crosslinking may be advisable. The introduction of crosslinks into a polymeric system is an established methodology for many types of polymers, and that methodology may be used to prepare crosslinked polymers of the present invention. For instance, divinyl-benzene may be introduced during the polymerization process, in order that the polymer contains crosslinks. Other suitable crosslinking agents are known in the art, and some of these have been identified elsewhere herein. Alternatively, the non-crosslinked polymer may be treated to introduce crosslinking by, for example, radiation. When present, the crosslinked residues typically constitute less than about 25% of the polymeric cationic polyelectrolyte residues. In various aspects, the crosslinked residues constitute less than about 20%, or 15%, or 10%, or 5%, or 2.5%, or 1%, or 0.1% of the residues that make-up the cationic polyelectrolyte.

The cationic polyelectrolyte will have a molecular weight, which may be described in terms of number average or weight average molecular weights. In various aspects of the invention, the molecular weight will be greater than about 1,000 (number average, i.e., Mₙ), or greater than about 5,000, or greater than about 10,000, or greater than about 50,000, or greater than about 100,000, or greater than about 500,000.

The cationic polyelectrolyte preferably contains quaternary ammonium groups. As used herein, a quaternary ammonium group has its ordinary and accustomed meaning, which is a central nitrogen atom bonded to four substituents other than hydrogen. The four substituents are each covalently bonded to the nitrogen. For instance, a protonated tertiary amine is not a quaternary ammonium group according to the present invention. The substituents bonded to the nitrogen are each organic groups, i.e., carbon-containing. Preferred substituents are hydrocarbons, which may be saturated or unsaturated. In one aspect, the hydrocarbon is aliphatic. In one aspect, the hydrocarbon is a saturated alkyl group, for example, a C1-C10 alkyl group (i e., an alkyl group having at least one and not more than 10 carbons), preferably a C1-C6 alkyl group, and in one aspect is a Cl alkyl group, i.e., methyl. In one aspect, the hydrocarbon is an unsaturated alkyl group, for example a C1-C10 unsaturated alkyl group, preferably a C1-C6 unsaturated alkyl group, and in one aspect is allyl.

In one aspect, the cationic polyelectrolyte contains residues of diallydialkylammonium, while in preferred aspects, at least 20%, at least 30%, at least 40%, at least 50%, at least 60%, at least 70%, at least 80%, at least 90%, at least 95% or 100% of the monomer residues of the cationic polyelectrolyte comprise dialkyldiallylammonium residues. In a preferred aspect, the residue providing the quaternary ammonium group is the diallyldimethylammonium group.

Because the cationic polyelectrolyte contains positively charged groups, the cationic polyelectrolyte will be in association with negatively charged species. In one aspect, the negatively charged species is a halide, e.g., bromide or chloride. Other negatively charged species with which the cationic polyelectrolyte may be in association include, without limitation, sulfate, phosphate, carbonate, nitrate, and organic carboxylates, e.g., acetate, propionate, etc.

In a preferred aspect, the cationic polyelectrolyte is poly(diallyldimethylammonium), also known as PDADMA. PDADMA is a material of. commerce, particularly in its form as the chloride salt (PDADMAC). For instance, PDADMAC may be obtained from Sigma, Milwaukee, Wis., U.S.A.; Polysciences, Warrington, Pa., U.S.A.; Qemi International, Inc., Kingwood, Tex., U.S.A.; and Ondeo Nalco Corporation, Naperville, Ill., U.S.A. However, the alkyl groups of a diallyldialkylammonium residue-containing cationic polyelectrolyte may be any C1-C6alkyl group, where a suitable monomer may be made in analogy with the know method of preparing poly(diallyidimethylammonium). Poly(diallyl dimethyl ammonium chloride) is also known as “PADMAC” or “polyDADMAC” or “PDADMAC”.

The synthesis of PADMAC involves two basic sequential steps: the formation of the monomer followed by the polymerization of the monomer. The monomer is usually formed by a reaction of a stoichiometric amount of dimethylamine and allyl chloride in an aqueous solution under alkaline conditions. In the presence of NaOH the monomer polymerizes resulting in a polymer having the following repeating unit:

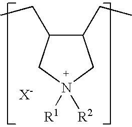

where R¹ and R² are linear or branched alkyl groups and X- is an anionic counterion, e.g., chloride or sulfate. The molecular weight of the monomeric unit is 161.5 and a typical molecular weight range for the PDADMAC is 50,000 amu to several million amu. There is one positive charge per monomeric unit.

The strong interactions that are observed between nucleic acids and cationic polyelectrolyte can be used to advantage according to the present invention. The present invention provides a method wherein oligonucleotides, which are either DNA- or RNA-based, as present in complex reaction mixtures, can be bound to a surface coated with a cationic polyelectrolyte, and then the reaction components other than the oligonucleotides can be washed away with water or aqueous solution, e.g., an ammonium salt solution. This leaves a surface having purified oligonucleotide in association with the cationic polyelectrolyte, where the bound oliognucleotide can be interrogated by, e.g., MALDI-MS. It has been discovered that the positively-charged cationic polyelectrolyte has an extremely high affinity for oligonucleotides, where this affinity can be used to advantage in preparing samples for MALDI-MS analysis. It has also been discovered that positively-charged cationic polyelectrolyte may have a relatively high affinity for oligonucleotides, in comparison to other materials commonly found in association with oligonucleotides.

The affinity of cationic polyelectrolyte and short oligonucleotides (6-mers to 50-mers, or 6-mers to 40-mers, or 6-mers to 30-mers) is so high that complexes of oligo and cationic polyelectrolyte cannot be easily dissociated by ionic detergents, high salt conditions, high divalent cation concentrations, or some conditions using organic solvents. That is, oligonucleotides are observed to bind to CP-coated solid supports or CP-coated surfaces in the presence of buffers, detergents, salts, divalent-cations and other typical reactions conditions employed in molecular-biology reactions. The oligonucleotides bind so tightly to the CP-coated surfaces that buffers, detergents, salts, divalent-cations and other typical reaction components can be washed from the surfaces while the oligonucleotides remain bound. A matrix (if necessary) can be applied to the solid support and the surface then can interrogated by a MALDI process. This method is extremely useful for molecular biology reactions that generate short oligonucleotides (i.e., 6-mers to 50-mers) as the readout or endpoint of the assay.

The matrix is an important feature of MALI-MS. Typically, analysis of nucleic acids by matrix-assisted laser desorption/ionization (MALDI) can be divided into two steps. The first step involves preparing the sample by mixing the sample to be analyzed with a molar excess of a chemical commonly referred to as the “matrix.” See, e.g., Wu et al. Rapid Commun. Mass Spectrom. 7:142-146 (1993). The primary purpose of the matrix is to promote ionization of the nucleic acid. Without the matrix, the nucleic acid molecule tends to fragment upon exposure to the laser energy, so that the mass and identity of the nucleic acid is difficult or impossible to determine. In general, the term “matrix” refers to a substance which absorbs radiation at a wavelength substantially corresponding to the pulse of laser energy used in the MALDI method, and where the matrix facilitates desorption and ionization of molecules. A matrix may be any one of several small, light-absorbing chemicals that may be mixed in solution with a nucleic acid in such a manner so that, upon drying on a solid support (e.g., a sample plate or a probe element), the crystalline matrix-embedded analyte molecules are successfully desorbed by laser irradiation and ionized from the solid phase crystals into the vapor phase and accelerate as intact molecular ions. The second step of the MALDI process involves desorption of the bulk portions of the solid sample by a short pulse of laser light.

While this is probably the most commonly employed approach to combining matrix and analyte prior to MALDI-MS analysis, other methods may be used. For instance, according to one aspect of the invention, the analyte-containing sample is added to (e.g., spotted onto) a coating of cationic polyelectrolyte, allowing the analyte (nucleic acid) to bind to the cationic polyelectrolyte. This spot is then washed in order to purify the nucleic acid. The spot is then treated with matrix (when the matrix is a liquid) or a solution of matrix (when the matrix is a solid). When the matrix is a solid, the spot should be allowed to dry in order to remove the solvent that was formerly used to dissolve the matrix in solution. Thereafter, this spot of nucleic acid and matrix can be subjected to MALDI-MS to provide a very strong signal due to the nucleic acid.

In another aspect the invention provides a method of performing an assay, comprising: (a) combining an analyte with a cationic polyelectrolyte to provide a mixture; (b) placing the mixture on a substrate to provide a sample; and (c) analyzing the sample by MALDI-MS. As in the other aspects of the invention, the cationic polyelectrolyte may, in one embodiment, comprise quaternary ammonium groups, e.g., the cationic polyelectrolyte may be PDADMAC. As mentioned above, MALDI-MS requires the presence of a matrix in the sample. In one aspect, the matrix is part of the mixture, while in another aspect the matrix is part of the sample. For instance, the matrix may be combined with analyte and cationic polyelectrolyte to form the mixture. The matrix may also be added to the sample after that sample has been placed on the solid support.

Accordingly, in one aspect, the present invention provides a solid support having a surface, where that surface is at least partially coated with a coating comprising cationic polyelectrolyte, where at least some of the cationic polyelectrolyte is in contact with nucleic acid and the nucleic acid is in contact with matrix. In a preferred embodiment, the solid support is a plate, e.g., a stainless steel plate, and the cationic polyelectrolyte either forms a continuous coating across all or a significant portion of the surface, or is spotted onto the surface in distinct regions. Nucleic acid and matrix is then located in distinct regions on the surface, so as to provide an array-type appearance. For example, the surface may be a 96-well plate, with cationic polyelectrolyte, nucleic acid and matrix located in one, and preferably more than one, of the wells. This array is then subjected to MALDI-MS, where the various regions are sequentially subjected to laser light, and the mass spectrum of the nucleic acid present in the spots is sequentially obtained.

To achieve its purpose, the matrix should meet one or more of the following criteria, and preferably meets many or all of these criteria. The matrix should be able to embed and isolate nucleic acid (e.g., by co-crystallization), it should be soluble in solvents compatible with nucleic acids, it should be stable under the vacuum used in MALDI, it should assist co-desorption of the nucleic acid upon laser irradiation, and it should promote ionization of the nucleic acid. In order to meet these criteria, the matrix should comprise a chromophore that strongly absorbs in the wavelength of light being emitted by the laser. For instance, if the laser is an ultraviolet laser, then the matrix should have a chromophore that absorbs in the ultraviolet region.

The following chemicals have been identified as suitable matrices for nucleic acids, where these as well as other suitable matrix chemicals known in the art may be used in the methods and compositions of the present invention: 6-aza-2-thiothymine (ATT), glycerol, 2,4,6-trihydroxyacetophenone (THAP), picolinic acid (PA), 3-hydroxy picolinic acid (HPA), 2,5-dihiydroxybenzoic acid, anthranilic acid, nicotinic acid, and salicylamide. Mixtures of these chemicals are also suitable. Typically, the matrix is a solid at room temperature. However, the matrix may be a liquid chemical, where suitable liquid matrices are substituted or unsubstituted: (1) alcohols, including: glycerol, 1,2- or 1,3-propane diol, 1,2-, 1,3- or 1,4-butane diol, triethanolamine; (2) carboxylic acids including: formic acid, lactic acid, acetic acid, propionic acid, butanoic acid, pentanoic acid, hexanoic acid and esters thereof; (3) primary or secondary amides including acetamide, propanamide, butanamide, pentanamide and hexanamide, whether branched or unbranched; (4) primary or secondary amines, including propylamine, butylamine, pentylamine, hexylamine, heptylamine, diethylamine and dipropylamine; (5) nitriles, hydrazine and hydrazide. These liquid matrices are particularly useful when the MALDI laser emits light in the infrared spectrum. It is reported that THAP works best for samples below 10 kDa while HPA and PA are more appropriate for oligonucleotides above 10 kDa. Acidic matrices, e.g., HPA, are preferred for single-stranded nucleic acids, while neutral matrices, e.g., glycerol and ATT, are preferred for double-stranded nucleic acids.

In one aspect, the solid support can be a plate that does not necessarily have indentations. However, in another embodiment, the solid support is a plate with indentations to receive sample. When the plate has indentations, then the indentations should have a coating of cationic polyelectrolyte. When the plate does not have indentations, then the cationic polyelectrolyte can simply be spread across the surface of the plate, or can be spotted into distinct regions. The plate can be made from any material that is not inconsistent with MALDI-MS analysis. Plates made from metal (e.g., stainless steel), inorganic materials (e.g., silicon) and organic materials (e.g., polyethylene) have all been successfully used in MALDI-MS applications. Accordingly, the following list of materials from which a plate may be prepared is exemplary only: steel (e.g., stainless-steel), glass fiber, glass, nylon 6/6, silicon, plastic, polyethylene, polypropylene, polyamide, or polyvinylidenedifluoride). The plate may be a composite, e.g., have a metallic surface consisting of steel, gold, silver, aluminum, copper, or the like, and have a different material underlying the surface, e.g., silicon. Such a coated plate is particularly useful, e.g., in those instances where polynucleotide is spotted onto a plate so as to form an array of spots, where some or all of the spots contain different polynucleotides.

The solid support need not be a plate. Any surface that can hold cationic polyelectrolyte, nucleic acid and matrix may be used according to the present invention. For instance, some MALDI-MS instruments are designed to receive a metallic probe, where the tip of the probe is coated with the analyte. These probes are another suitable solid support.

The plate can be further coated at least partially by an organic matrix. As mentioned above, a matrix that absorbs radiation from an incident UV or IR laser is often applied to a MALDI plate, where this energy is transferred from the matrix to the sample resulting in desorption of the sample into the gas phase with subsequent ionization and minimal fragmentation. In one embodiment, the present invention provides a plate that is coated with both a cationic polyelectrolyte and an energy-transferring matrix. Such matrix chemicals are well known in the art of MALDI, and include, e.g., 3-hydroxypicolinic acid and trifluoroacetic acid, as described above. The plate with a polynucleotide array can be subjected to analysis, e.g., by MALDI mass spectrometry, where the polynucleotides on the polymeric coating are subjected to laser light and injected into the mass spectrometer.

In another aspect, the solid support can be a modified plate having distinct indented surfaces or wells. The plate may optionally have 10 to 1,000 wells, e.g., may have wells arranged as in standard 96- and 384-well plates. For instance, the plate may be a microtiter plate, made of ceramic, glass, stainless-steel, or plastic (e.g., polyethylene, polypropylene, polyamide, polyvinylidenedifluoride). Such microtiter plates are commercially available having various numbers of indented surfaces, which are also called wells. A suitable plate useful in the present invention may have 96 wells or 384 wells, or other suitable number of wells. Microtiter plates are available from a variety of vendors including: Applied Biosystems, Foster City, Calif., Applied Scientific, South San Francisco, Calif., and Brinkmann Instruments, Inc., Westbury, N.Y. Stainless-steel plates with 2 mm diameter spots or wells that are specific to MALDI-MS are also available from vendors such as Micromass.

The surface of the above plate is at least partially coated with a coating comprising a cationic polyelectrolyte. The polyelectrolyte preferably contains multiple quaternary ammonium-containing residues, e.g., diallyidialkylammonium residues, where preferably at least 5% of the monomer residues include quaternary ammonium groups. A preferred embodiment would employ PDADMAC as the cationic polyelectrolyte. Various methods are available to modify the plate so that it contains cationic polyelectrolyte in the wells of the plate. For instance, a solution of the cationic polyelectrolyte may be prepared, and then the solution coated onto the wells. Upon evaporation of the solvent, the well will contain a coating of the cationic polyelectrolyte, such that a modified plate results.

Alternatively, the plates may contain a functionality localized to the wells of the plate. For instance, the wells of a plate may be modified to contain one member of a binding pair, for instance avidin or strepavidin or biotin. Such plates are commercially available Promega, Madison, Wis., Millipore, Bedford, Mass., and Sigma, Milwaukee, Wis. The cationic polyelectrolyte is modified to contain the other member of the binding pair e.g., biotin or avidin or strepavidin. A solution of the modified cationic polyelectrolyte is added to the modified plate and the two halves of the ligand pair bind, so as to couple the polymer to the plate in a covalent manner.

In one aspect, the solid support can be balls or beads where the surfaces are at least partially coated with a coating comprising cationic polyelectrolyte (CP). The cationic polyelectrolyte preferably contains multiple quaternary ammonium-containing residues, e.g., diallyldialkylammonium residues, where preferably at least 5% of the monomer residues include quaternary ammonium groups. A preferred embodiment would employ PDADMAC as the cationic polyelectrolyte. The balls or beads may comprise, for example, steel (e.g., stainless-steel), glass, silica, organic polymer, magnetic material, Sephadex or Sephasrose, cellulose. Such coated balls or beads are useful, e.g., in those instances where polynucleotide is extracted from a cell lysate or reaction. The CP/polynucleotide coated balls or beads can be further coated at least partially by an organic matrix that transfers energy under MALDI conditions, where suitable matrices have been described herein. The balls or beads with a polynucleotide/CP complex coating can be subjected to analysis, e.g., by MALDI mass spectrometry.

In another aspect, polynucleotides can be analyzed from a simple or complex mixture by a MALDI-MS method. The present invention provides a method of performing a polynucleotide assay where the method includes: (a) providing a solid support comprising a surface, the surface at least partially coated with a cationic polyelectrolyte; (b) applying a polynucleotide solution to the coated surface; (c) allowing the polynucleotide solution to interact with the polymer; (d) washing the polymer/polynucleotide complex to remove at least some components of the sample; (e) applying an organic matrix solution; and (f) measuring the polynucleotide by MALDI-MS.

In this method, the cationic polyelectrolyte comprises a polymer preferably having multiple quaternary ammonium-containing residues, e.g., diallydialkylammonium residues, where preferably at least 5% of the monomer residues include quaternary ammonium groups. A preferred embodiment would employ PDADMAC as the polymer. Preferred solid supports include: 1) beads comprising silica, glass, magnetic material, Sephadex or Sepharose, cellulose, or stainless-steel; 2) capillary tubes, which may be made from the same materials from which the beads are prepared; 3) plates comprising steel, glass fiber, glass, nylon 6/6, silicon, or plastics such as polyethylene, polypropylene, polyamide, and polyvinylidenedifluoride; 4) multiwell plates comprising plastics such as polyethylene, polypropylene, polyamide, polyvinylidenedifluoride, steel and silicon, 5) a membrane comprising a plastic such as polyethylene, polypropylene, polyamide, or polyvinylidenedifluoride; 6) pins or an array of pins suitable for combinatorial synthesis or analysis; or 7) beads in pits of otherwise flat surfaces such as wafers (e.g., silicon wafers) with or without filter plates.

In one aspect of the invention, a solution containing one or more polynucleotides is applied to a CP-coated surface and allowed to interact with the CP for a time appropriate for the polynucleotide to adhere to the polymer. This time is typically on the order of 1 second to 24 hours. After the appropriate time has passed, the polymer/polynucleotide complex is washed with water, preferably purified water, and optionally additionally washed with an ammonium salt solution. As discussed above, the polynucleotide has a relatively high affinity for the CP coated surface such that other components of the polynucleotide solution, such as salts, detergents, buffers and the like do not adhere and are washed away in the water washing. The ammonium salt wash replaces any ions associated with the phosphate groups on the polynucleotide. After drying the polymer/polynucleotide complex, an organic matrix is applied to the complex. In a preferred embodiment, the organic matrix would comprise 3-hydroxypicolinic acid and trifluoroacetic acid. The organic matrix solvent, if present, is then evaporated and the polynucleotide(s) analyzed by MALDI-MS.

The present invention also provides a method whereby polynucleotides can be analyzed from a simple or complex mixture by MALDI-MS. Thus, the present invention provides a method of performing a polynucleotide assay where the method includes: (a) providing a solid support comprising a surface, the surface at least partially coated with a cationic polyelectrolyte; (b) applying a polynucleotide solution to the coated surface; (c) allowing the polynucleotide solution to interact with the polymer; (d) washing the polymer/polynucleotide complex; and (e) measuring the polynucleotide by MALDI-MS. In a preferred embodiment, matrix is added to the location where the polynucleotide is located on the cationic polyelectrolyte. In this method, the cationic polyelectrolyte is preferably has multiple quaternary ammonium-containing residues, e.g., diallydialkylammonium residues, where preferably at least 5% of the monomer residues include quaternary ammonium groups. A preferred embodiment employs PDADMAC as the cationic polyelectrolyte. Exemplary solid supports include: 1) beads comprising silica, glass, magnetic material, Sephadex or Sepharose, cellulose, or stainless-steel; 2) capillary; 3) plates comprising steel, glass fiber, glass, nylon 6/6, silicon, or plastics such as polyethylene, polypropylene, polyamide, and polyvinylidenedifluoride; 4) multiwell plates comprising plastics such as polyethylene, polypropylene, polyamide, and polyvinylidenedifluoride, steel and silicon, 5) a membrane comprising a plastic such as polyethylene, polypropylene, polyamide, or polyvinylidenedifluoride; 6) pins or an array of pins suitable for combinatorial synthesis or analysis; or 7) beads in pits of flat surfaces such as wafers (e.g., silicon wafers) with or without filter plates.

In another aspect, the present invention provides a dipstick with a coating of a cationic polyelectrolyte, where the cationic polyelectrolyte preferably comprises monomer residues, and at least 5% of the monomer residues include quaternary ammonium groups. The polymer-coated dipstick may be used for isolating polynucleotides from a mixture, such as PCR product, cell lysate, or any other solution containing polynucleotide. The substrate is made of e.g., nylon or another polymer, nitrocellulose, or glass. A preferred embodiment would employ PDADMAC as the polymeric coating. Various methods may be used in order to modify the substrate so that it is covered, at least partially, with cationic polyelectrolyte. For instance, a solution of the cationic polyelectrolyte may be prepared followed by coating the substrate with the solution. Upon evaporation of the solvent, the substrate will have a coating of the cationic polyelectrolyte, such that a modified substrate results. The polynucleotide(s) is isolated from the solution by inserting the dipstick into the polynucleotide containing solution or, alternatively, coating the polynucleotide containing solution onto the substrate. The solution and the substrate are allowed to interact for a time sufficient (1 second to 24 hours) to allow the polynucleotides to adhere to the cationic polyelectrolyte. Adherence between the polyelectrolyte and polynucleotide is an inherent reaction between to the two entities. The substrate is either removed from the solution or the solution coating removed. At least some of the polynucleotide is extracted from the solution through adherence to the coating of the substrate. Following contact with the polynucleotide solution, the substrate may or may not require washing to eliminate residual solution and its components.

In another aspect, a filter coated with cationic polyelectrolyte may be used for isolating polynucleotides from a mixture. The filter is a porous matrix, where the matrix is coated with a cationic polyelectrolyte that preferably includes multiple quaternary ammonium-containing residues, e.g., diallyidialkylammonium residues, where preferably at least 5% of the monomer residues include quaternary ammonium groups. A preferred embodiment would employ PDADMAC as the cationic polyelecytrolyte on the filter. The filter may be made of e.g., nylon or another polymer. Various methods can be used to modify the filter so that it is covered, at least partially, with cationic polyelectrolyte. For instance, one may prepare a solution of the cationic polyelectrolyte, and then coat the filter with the solution. Upon evaporation of the solvent, the filter will have a coating of the polymer, such that a modified substrate results. Alternatively, one may purchase a filter that contains functionality localized on the surface of the substrate. Such a filter can be purchased at Millipore, Bedford, MA. The filter may then be reacted with a cationic polyelectrolyte as described above, so as to couple the polyelectrolyte to the filter in a covalent manner. As a third example, the filter may be modified to contain one member of a binding pair, for instance avidin or strepavidin and biotin. The cationic polyelectrolyte is then modified to contain the other member of the binding pair. Upon addition of a solution of the binding partner-modified polymer to the filter, the cationic polyelectrolyte will become attached to the filter. The filter may be used for isolating polynucleotides from a mixture, such as PCR product, cell lysate, or any other solution containing polynucleotide. The polynucleotide is isolated from the polynucleotide-containing solution by passing the solution through the modified filter. Thereafter, at least some of the polynucleotide is separated from the solution through adherence to the cationic polyelectrolyte on the filter. Following passage of the solution, the filter may or may not require washing to remove residual solution and its components.

These various devices containing cationic polyelectrolyte may be used, according to one aspect of the invention, in analyzing for the present of a biomolecule or other organic molecule in an environment. These and other suitable devices, as well as methods of using these and other suitable devices, are very valuable in detecting undesirable materials in an environment, e.g., a pathogen and/or a toxin. Many pathogens produce toxins that are harmful, and even lethal, to a host organism. Toxins produced by pathogens can be classified into two general categories, exotoxins and endotoxins. Exotoxins are generally proteins or polypeptides. These toxins, which are secreted by the pathogen, can travel within the host and cause damage in regions of the host far removed from the infection site. Symptoms associated with exotoxins include hemolysis, systemic shock, destruction of leukocytes, vomiting, paralysis and diarrhea. Enterotoxins are exotoxins which act on the small intestine and cause massive secretion of fluid into the intestinal lumen, leading to diarrhea. Enterotoxins are produced by a variety of bacteria, including the food-poisoning organisms Staphylococcus aureus, Clostridium perfringens, and Bacillus cereus, and the intestinal pathogens Vibrio cholerae, Escherichia coli, and Salmonella enteritidis. Endotoxins are lipopolysaccharides/lipoproteins found in the outer layer of the cell walls of gram-negative bacteria. These lipopolysaccharides are bound to the cell membrane and are released upon cytolysis. Symptoms associated with the release of endotoxins include fever, diarrhea and vomiting. Although endotoxins are less toxic than exotoxins, large doses of endotoxins can cause death, generally through hemorrhagic shock and tissue necrosis. Examples of bacteria which produce endotoxins include bacteria of the genera Escherichia, Shigella, and especially Salmonella. A microbial toxin is an endotoxin or exotoxin produced by a microorganism, such as a bacterium, a fungus or a protozoan, where either endotoxins or exotoxins may be detected according to the present invention. Other types of organic materials that may be detected according to the present invention include acidic compounds, neutral compounds, basic compounds, nucleic acids, proteins, and polysaccharides, where biohazardous organic compounds are of particular interest.

Thus, in one aspect the present invention provides a method for environmental analysis comprising: (a) contacting an environment with a solid support comprising a surface, the surface being at least partially coated with a cationic polyelectrolyte, the cationic polyelectrolyte being contacted with the environment for a time sufficient for the catonic polyelectrolyte to absorb one or more organic compounds from the environment so as to provide a contaminated surface; and (b) analyzing the contaminated surface by MALDI-MS to provide an identification of the one or more organic compounds present in the environment.

In another aspect, the present invention provides a method for detecting the presence or absence of a biomolecule in an environment, comprising: (a) exposing cationic polyelectrolyte to an environment, where the environment may or may not contain an organic biomolecule; (b) allowing the cationic polyelectrolyte to interact with the environment so as to absorb a biomolecule from the environment in the event a biomolecule is present in the environment, thereby providing a potentially contaminated cationic polyelectrolyte; and (c) analyzing the potentially contaminated cationic polyelectrolyte by MALDI-MS to determine whether an organic biomolecule was present in the environment.

In the method for environmental analysis and the method for detecting the presence or absence of a biomolecule in an environment, in various optional embodiments: the organic compound/biomolecule is selected from the compounds described above, including amino acid, saccharide and nucleotide; or a high molecular weight material having a plurality of residues selected from amino acid (e.g., a polypeptide), saccharide (e.g., a polysaccharide) and nucleotide (e.g., a polynucleotide) residues. In one aspect of the invention, the organic compound/biomolecule is indicative of, or presents, a health hazard to animals, particularly mammals. The present invention is particularly useful in passively sampling air and water, where organic compounds present in these environments are absorbed onto the cationic polyelectrolyte to provide “contaminated” cationic polyelectrolyte. After an appropriate period of time, the potentially contaminated cationic polyelectrolyte is treated with a matrix and then subjected to MALDI-MS in order to identify the organic compounds in the environment.

The cationic polyelectrolyte may be part of a composite structure. Many if not most cationic polyelectrolytes do not have sufficient structural integrity to exist as a plate or other structure that may be stabling placed into an environment. While it is possible to place a powdered or other particulate cationic polyelectrolyte in a container, and place that container in an atmosphere, the same is not possible for an aqueous environment. Accordingly, at least when the cationic polyelectrolyte is to be placed in an aqueous environment, it is preferred to have the polyelectrolte as part of a composite structure.

In one aspect, the composite structure is a solid support having a cationic polyelectrolyte coating. The solid support may be made from any suitable material, where the durability, flexibility, cost and strength of the solid support should be selected with a view to the environment into which the composite will be placed. Exemplary solid supports have been disclosed elsewhere herein, and include metal, plastics and glass. Stainless steel is a preferred solid support because stainless steel is widely used as a substrate for samples in MALDI-MS analysis. Stainless steel is also robust under many environmental conditions.

The cationic polyelectrolyte may be, for example, any of the cationic polyelectrolytes disclosed elsewhere herein. Cationic polyelectrolytes having quaternary ammonium groups, and PDADMAC in particular, are preferred cationic polyelectrolytes.

The cationic polyelectrolyte should be positioned in an environment for a sufficient time to allow the polyelectrolyte to absorb organic molecules from the environment. This time will vary depending on many factors, including the specific organic molecules, the concentration of the organic molecules in the environment, the rate at which the organic molecules impact the cationic polyelectrolyte, the identify of the cationic polyelectrolyte, and the strength of the signal desired in the mass spectrometer, to name a few. This time will be selected in accordance with the purpose of the analysis, e.g., the polyelectrolyte may be analyzed and replaced on a weekly basis if it is desired to know, for each weekly period, the amount of biomolecule in the air or other environment being studied.

In other aspects, the present invention provides kits for analyzing organic molecules and biomolecules in an environment. For example, the invention provides a kit for detecting the presence of an organic biomolecule in an air supply, comprising: (a) a solid support comprising a cationic polyelectrolyte; and (b) a means for securing the solid support in the air supply. As another examples, the invention provides a kit for detecting the presence of an organic biomolecule in a water supply, comprising: (a) a solid support comprising a cationic polyelectrolyte; and (b) a means for securing the solid support in the water supply. The these kits, in one aspect, the cationic polyelectrolyte comprises quaternary ammonium groups, e.g., the cationic polyelectrolyte is PDADMAC.

The means for securing the solid support in the environment may be, for example, a clip, a clamp, a magnet, tape such as adhesive tape, transparent tape, electrical tape, Velcro-type fastners, glue, adhesive, screws, bolts, nails, or the like.

The following summarizes some of the preferred embodiments of the present invention. In one such embodiment, (embodiment Al), the present invention provides a plate comprising a surface, where the surface is at least partially coated with a coating comprising a cationic polyelectrolyte. Optionally, the plate may have wells, e.g., 10 to 1,000 wells, although plates having wells numbering in s multiple of eight (i.e., 8, 16, 24 . . . 96, 104, etc.) are preferred because much of the technology of today utilizes such plates. In one embodiment, the coating of the plate is in contact with polynucleotide, e.g., is in contact with an array of polynucleotides. This will typically be formed after the plate has been contacted with an analyte.

The analysis of nucleic acids by MALDI-MS, using samples that contain both nucleic acid and CP, is an important aspect of the present invention. Accordingly, in one embodiment (embodiment A2), the present invention provides a plate suitable for supporting a sample in a MALDI analysis, the plate comprising a coating of cationic polyelectrolyte. This plate will be very useful in accepting nucleic acid analyte, and thereafter optionally purifying the nucleic acid away from materials that do not so readily adhere to the CP. The plate may be formed, at least partially, or wholly, from stainless steel. In addition to CP, the plate may optionally contain a matrix coating, where the matrix coating is able to transfer energy from an energy source to a sample on the plate, as occurs during MALDI-MS analysis. Upon being contacted with an analyte, this plate will further include a biomolecule, where a polynucleotide is a preferred biomolcule to have located on the plate. In one aspect, the polynucleotide is relatively short, i.e., has 5-50 nucleotides. However, in another aspect, the polynucleotide is somewhat longer, i.e., has 50-100 nucleotides.

Related to MALDI-MS analysis, in a related embodiment (embodiment A3), the present invention provides a composition comprising a cationic polyelectrolyte and a MALDI matrix. This composition is very convenient for combining with an analyte to be analyzed by MALDI-MS. Upon such combination, the present invention provides a composition that comprises a cationic polyelectrolyte, a MALDI matrix, and an analyte, preferably a biomolecule, e.g., a nucleic acid, a protein, or a mixture of nucleic acid and protein. Particularly when the analyte comes from an assay, the composition may further include water. In some instances, the analyte may come from the eluent of a chromatography separation, and so in this and other instances, the composition of CP and MALDI matrix may comprise an organic solvent, e.g., acetonitrile.

In a related embodiment (embodiment A4), the present invention provides a sample suitable for MALDI analysis. This sample includes a solid, support that has a surface, where the surface is at least partially covered with a coating, and the coating comprises a cationic polyelctrolyte. The sample also includes a MALDI matrix, and further includes nucleic acid. The solid support may be made from any material that is suitably used, and amenable, in MALDI-MS analysis. Such materials include stainless steel, nickel, and platinum. However, material might also be an organic polymer.

In another embodiment (embodiment A5), the present invention provides a solid support comprising a surface, the surface at least partially coated with a cationic polyelectrolyte and an organic matrix. Optionally, the support is in the form of a plate, and optionally, the support is in the form of a multiwell plate, e.g., a plate having 10 to 1,000 wells. Alternatively, the support is in the form of beads, e.g., beads made from silica, glass, a magnetic material, Sephadex, Sepharose, cellulose, stainless-steel or organic polymer. Optionally, the support is in the form of an array of pins.

In another embodiment (embodiment A6), the present invention provides a dipstick comprising a surface, where the surface is at least partially coated with a coating comprising a cationic polyelectrolyte. As with the plate discussed above, optionally, the cationic polyelectrolyte comprises a plurality of quaternary ammonium groups, where optionally the cationic polyelectrolyte comprises the polymerization product of diallyidimethylammonium in association with a counterion, where a preferred coating of this type contains poly(dimethyidiallyl ammonium) chloride (PDADMAC).

In another embodiment (embodiment A7), the present invention provides a filter comprising a porous matrix, where the matrix comprises a surface, and the surface is at least partially coated with a coating comprising a cationic polyelectrolyte.

In another embodiment (embodiment A8), the invention provides a method for performing an assay, which entails: (a) providing a plate having wells with distinct indented surfaces, the surfaces coated with a cationic polyelectrolyte; (b) performing a nucleotide extension assay in a well of (a); and (c) detecting the presence or absence of extension product.

In another embodiment (embodiment A9) the present invention provides another method for performing an assay. This method includes: (a) providing a solid support comprising a surface, the surface at least partially coated with a cationic polyelectrolyte; (b) applying a polynucleotide solution to the coated surface; (c) allowing the polynucleotide solution to interact with the polymer; (d) washing the polymer/polynucleotide complex; (e) applying an organic matrix solution; and (f) measuring the polynucleotide by MALDI-MS. Optionally, the polynucleotide solution will contain a plurality of distinct polynucleotides, where the assay will provide information about those polynucleotides. Optionally, the wash consists of water washings and at least one washing with an ammonium salt solution.

In another embodiment (embodiment A10), the invention provides yet another method of performing an assay. This method includes: (a) providing a solid support comprising a surface, the surface at least partially coated with a cationic polyelectrolyte; (b) applying a polynucleotide solution to the coated surface; (c) allowing the polynucleotide solution to interact with the polymer; (d) washing the polymer/polynucleotide complex; and (e) measuring the polynucleotide by MALDI-MS. This is a particularly efficient method to analyze polynucleotides that are present in complex mixtures, because the polynucleotide will preferentially adhere to the polymer.

In another embodiment (embodiment A11), the present invention provides yet another way of performing an assay. This method includes: (a) combining an analyte with a cationic polyelectrolyte to provide a mixture; (b) placing the mixture on a substrate to provide a sample; (c) analyzing the sample by MALDI-MS. This method is convenient in that the CP may be added to a mixture containing analyte of interest, and then the mixture is placed on a probe or other surface amenable to MALDA-MS analysis. Optionally, it may be desired to wash the mixture after it has been placed on the surface, in order to remove contaminants. In a preferred but not required embodiment, matrix will be present in the mixture present on the probe, where the matrix may be added to the analyte along with the polymer, or the matrix may be added later, i.e., after the polymer and analyte are placed on the surface.

In another embodiment (embodiment A12), the present invention provides a method of isolating polynucleotides. This method comprises, in sequence: (a) providing a solution comprising polynucleotides; (b) inserting a substrate into the solution, the substrate at least partially coated with a cationic polyelectrolyte; (c) allowing the polynucleotides to adhere to the cationic polyelectrolyte; and (d) removing the substrate and at least some of the polynucleotides from the solution. In this way, the polynucleotide has been isolated from materials it was previously in association with.

The present invention, in a related embodiment (embodiment A13) provides a method for purifying a nucleic acid. This method includes: (a) providing a mixture comprising nucleic acid and non-nucleic acid; (b) providing a solid support comprising a coating comprising cationic polyelectrolyte (e.g., a plate or dipstick); (c) contacting the mixture with the cationic polyelectrolyte under conditions where nucleic acid adheres to the cationic polyelectrolyte, to provide adhered nucleic acid; (d) separating adhered nucleic acid from at least some of the non-nucleic acid; and (e) characterizing the adhered nucleic acid. This method is very convenient for purifying samples prepared by an assay, where the non-nucleic acid may be or include protein, and adhered nucleic acid is separated from protein prior to the characterization of the adhered nucleic acid. Regardless of what the non-nucleic acid is, in a preferred embodiment the adhered nucleic acid is separated from non-nucleic acid by washing the coating of adhered nucleic acid with water. Thereafter, the adhered nucleic acid may optionally be characterized by MALDI-MS. In this case, the adhered nucleic acid is preferably contacted with matrix prior to being characterized by MALDI-MS.

In each of embodiments Al, A2, A3, 4, A5, A6, A7, A8, A9, A10, A11, A12 and A13, and elsewhere herein, the term “a” refers to one or more. Also, for each of these thirteen embodiments, the cationic polyelectrolyte optionally comprises a plurality of quaternary ammonium groups, where optionally the cationic polyelectrolyte comprises the polymerization product of diallyidimethylammonium in association with a counterion, where a preferred coating of this type contains poly(dimethyldiallyl ammonium) chloride (PDADMAC). Also, for each of these embodiments, the surface coated with CP may be at least partially formed from steel, glass fiber, glass, nylon 6/6, silicon, polyethylene, polypropylene, polyamide, polyvinylidenedifluoride, gold, silver, aluminum, copper, and plastic.

The devices and methods of the present invention are particularly useful in environmental analysis, that is, determining if something is present (or absent) in an environment. To this end, in one embodiment (A14), the present invention provides a method that include: (a) contacting an environment with a solid support comprising a surface, the surface being at least partially coated with a cationic polyelectrolyte. The cationic polyelectrolyte is contacted with the environment for a time sufficient for the cationic polyelectrolyte to absorb one or more organic compounds from the environment so as to provide a contaminated surface. (b) analyzing the contaminated surface by MALDI-MS to provide an identification of the one or more organic compounds present in the environment.

In a related embodiment (embodiment A15), the present invention provides a method for detecting the presence or absence of a biomolecule in an environment. This method includes: (a) exposing cationic polyelectrolyte to an environment, where the environment may or may not contain an organic biomolecule; (b) allowing the cationic polyelectrolyte to interact with the environment so as to absorb a biomolecule from the environment in the event a biomolecule is present in the environment, thereby providing a potentially contaminated cationic polyelectrolyte; and (c) analyzing the potentially contaminated cationic polyelectrolyte by MALDI-MS to determine whether an organic biomolecule was present in the environment.

In optional aspects of embodiments A14 and A15, the environment is an atmosphere, or the environment is a water supply. In other optional aspects, the organic compound or biomolecule is an amino acid, or is a saccharide or is a nucleotide, or contains a plurality of amino acid residues, or contains a plurality of saccharide residues, or contains a plurality of nucleotide residues, or is a polypeptide, or is a polysaccharide, or is a polynucleotide. In a preferred aspect, the organic compound/biomolcule is indicative of a health hazard in the environment. For example, the compound/biomolecule may be indicative of a pathogen.

In each of embodiments A14 and A15, the cationic polyelectrolyte optionally comprises a plurality of quaternary ammonium groups, where optionally the cationic polyelectrolyte comprises the polymerization product of diallyidimethylammonium in association with a counterion, where a preferred coating of this type contains poly(dimethyidiallyl ammonium) chloride (PDADMAC). Also, for each of these embodiments, the surface coated with CP may be at least partially formed from steel, glass fiber, glass, nylon 6/6, silicon, polyethylene, polypropylene, polyamide, polyvinylidenedifluoride, gold, silver, aluminum, copper, and plastic. For instance, the cationic polyelectrolyte may be part of a composite structure, e.g., a solid support having a coating of cationic polyelectrolyte on the solid support.

In related embodiments, the present invention provides kits that may be used for environmental analysis. The kits will have certain similar elements, e.g., a surface coated, at least in part, with cationic polyelectrolyte. However, the kits will typically be tailored to a specific environment. For instance, in one embodiment (embodiment A16), the prevent invention provides a kit for detecting the presence (or absence) of an organic biomolecule in an air supply. This kit includes (a) a solid support comprising a cationic polyelectrolyte; and (b) a means for securing the solid support in the air supply. For example, the solid support may be a plate with an adhesive backing, where the plate can be adhered to the wall of a building, and the outward facing side of the plate has a coating of CP. In a related embodiment (embodiment A17), the present invention provides a kit for detecting the presence of an organic biomolecule in a water supply. This kit includes: (a) a solid support comprising a cationic polyelectrolyte; and (b) a means for securing the solid support in the water supply.

The following examples are intended to illustrate various features and aspects of the present invention, and should not be construed as a limitation thereon.

### EXAMPLES

### Example 1

### Analysis of Oligonucleotides Using PDADMAC-Coated Maldi Card.

Procedure

A stainless-steel MALDI card was obtained from Micromass LTD (Manchester UK). The card was designed to accommodate 96-wells or “reading spots.” The top 4 rows (48 wells in all (A1-D12)) were treated with 0.1% w/v PDADMAC in 0.2×SSC, pH 7.6. Each spot received approximately ½ μL of the PDADMAC solution. The spots were allowed to dry, and then the card was washed with 10 mL of HPLC-grade water (Fisher). The card was the dried on top of a MJ thermocycler at 50° C. for 5 minutes.

An oligonucleotide of 3643 daltons (having a sequence of 5′-TCCAGTCGTAGG-3′ and molecular weight=3643 amu) was placed in the first 6 wells as well as 6 wells on the card that had not been treated with PDADMAC. The oligonucleotide concentration was 1 picomole per microliter (1 μM). Approximately 1 μL was added to the wells. The solution was incubated for 5 minutes at 28° C. The solution was decanted by aspiration. All of the wells were washed with HPLC-grade water three times, and then twice with 10 mg/mL ammonium citrate. The card was then dried at 50° C. and the matrix was added to the spots. The matrix was composed of 25 mg/mL 3-hydroxypicolinic acid (Fluka, St. Louis, Mo.) in 50:50 mixture of acetonitrile (ACN)/water and 0.05% trifluoroacetic acid (TFA). Approximately ½ μL was added per spot. The spots were dried for 5 minutes at 37° C.

The MALDI conditions were as follows: Signal sensitivity=100.0; Function 1: polarity=LDI+; Source voltage (v)=15014; Pulse voltage=1750; MCP detector voltage=1950; Laser energy=20%; Matrix suppression (amu)=493.2; TLF delay (ns)=500.0; scans in function=10; cycle time (secs)=0.600; Ionization mode=LD+; retention window (minutes)=0.00-60.00; Data type=accuruate mass; Function type=Maldi TOF; mass range=1000-2000. Instrument calibration: MS1 static=none; MS1 scanning=none; MS1 scan speed=none; MS2 static=none; MS1 scanning=none; MS2 scan speed=none. Coefficients: MS1 static=none; MS2 static=none; function 1=0.000040788799*x2+1.001781806596*x+0.230258255211, root mass. Sample period (ns) =0.5; signal sensitivity=100.0.

Results:

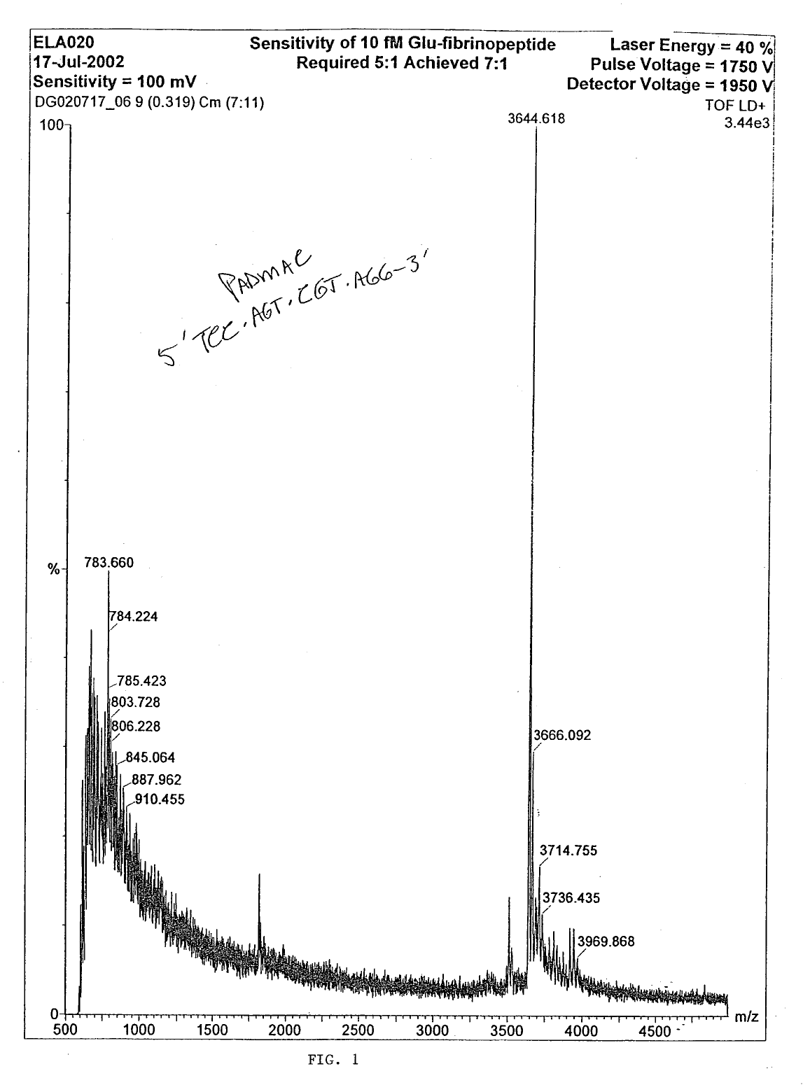

In the wells, that contained the oligonucleotide, an ion at 3644.618 was strongly observed (see FIG. 1). A minor Na+adduct was observed at 3666.092. There was no signal in the wells containing PDADMAC alone and no signal in the wells which were spotted in the absence of PDADMAC.

### Example 2

### Detection of A Genotyping Reaction Using PDADMAC Coated Cards

This example describes the amplification of a fragment containing a single nucleotide polymorphism (SNP) and its subsequent detection with a MALDI/TOF mass spectrometry system. Procedure:

The nucleic acids involved in this assay may be described as follows:

```
tgacatcattggctgagtctttcga
                                              gaacacGtgata
tgacatcattggctgagtctttcgaacacGtgatagaaggactcttggatgaggaccagaa
actgtagtaaccgactcagaaagcttgtgCactatcttcctgagaacctactcctggtctt
                                              cttgtgCactat
                                                                  atcttcctgagaacctactcctggtctt
```

where

5′-tgacatcattggctgagtctttcga-3′ is the forward primer

3′-atcttcctgagaacctactcctggtctt-5′ is the reverse primer and

5′-gaacacgtgata-3′ is the forward end product for the abundant allele

3′-cttgtgCactat-5′ is the reverse end product for the abundant allele and

5′- tgacatcattggctgagtctttcgaacacGtgatagaaggactcttggatgaggaccagaa-3′ is the forward initial amplicon

3′-actgtagtaaccgactcagaaagcttgtgCactatcttcctgagaacctactcctgqtctt-5′ is the reverse initial amplicon

After the initial PCR amplification, the amplicon will be over 50 nucleotides in length. In this example the SNP is biallelic and is a G/A change (the abundant allele is G and the rare allele is A).

TABLE 1

Particulars about the assay are presented in Table 1.

| | |
|---|---|
| Snp_id | 89 (BDNF_V66M) |

| | |
|---|---|
| Plus_primer_seq | Tgacatcattggctgagtctttcga |
| Plus_primer_location | 72 |
| Plus_primer_tm | 64.50 |
| Minus_primer_seq | Ttctggtcctcatccaagagtccttcta |
| Minus_primer_location | 132 |
| Minus_primer_tm | 64.50 |
| Plus_frag_seq | Gaacacgtgata |
| Plus_frag_location | 95 |
| Minus_frag_seq | Tatcacgtgttc |
| Minus_frag_location | 95 |
| Plus_per_seq | tgacatcattggctgagtctttcgaacacgtgatagaaggactcttggatgaggaccagaa |
| Plus_per_loction | 72 |
| Plus_per_tm | 68.90 |
| Notes | brain-derived growth factor SNP |
| Alleles | G, A |
| Min Mass Diff | 2.02 |

The mass/charge calculations for all possible alleles are as set forth in Table 2 for electrospray in negative mode:

TABLE 2

| | | | |
|---|---|---|---|
| Allele A | Plus | gaacacAtgata | 3742.48 |
| | Minus | tatcaTgtgttc | 3706.44 |
| Allele C | Plus | gaacacCtgata | 3718.46 |
| | Minus | tatcaGgtgttc | 3731.45 |
| Allele G | Plus | gaacacGtgata | 3758.48 |
| | Minus | tatcaCgtgttc | 3691.43 |
| Allele T | Plus | gaacacTtgata | 3733.47 |
| | Minus | tatcaAgtgttc | 3715.45 |

Other fragments produced by the assay:

| | | | | |
|---|---|---|---|---|
| Plus Strand | 5′ frag | Plus | tgacatcattggctgagtctttc | 7020.68 |
| Minus Strand | 5′ frag | Minus | ttctggtcctcatccaagagtccttc | 7848.23 |

An initial amplification step is performed to incorporate the recognition site for the N.BstNBI nicking enzyme into the DNA fragment on either side of the SNP site. Either of the following two procedures may be used to achieve this amplification:

(1) The 50 μL polymerase chain reactions were composed of 5-25 ng genomic DNA, 0.4 μM each forward and reverse primers, 10 mM Tris pH 8.3, 50 mM KCl, 1.5-4 mM MgCl₂, 200 μM each dNTP, 1 Unit DNA Polymerase (MasterAmp™ Taq DNA Polymerase from Epicentre Technologies, Madison Wis. or Vent exo-Polymerase New England BioLabs, Beverly Mass.). Thermocycling conditions were as follows: 95° C. for 3 minutes initial denaturation; 30 cycles of 92° C. for 40 seconds, 60° C. for 30 seconds, 72° C. for 30 seconds. A MJ Research PTC-100 thermocycler (MJ Research, Watertown, Mass.) was used for all PCR reactions. Primers were purchased from MWG Biotech (High Point, N.C.); or

(2) The following PCR mixture was assembled at 4° C. A total reaction volume of 50 μL per well in a 96-well microtiter plate (Fisher, St Louis Mo.) containing the following: 0.4 μM forward and reverse primers, 400 μM dNTPs, 5 nanogram of genomic DNA (Coriell DNAs from Coriell Institute, Bethesda Md.), 1× PCR buffer (EpiCentre Technologies, Austin Tex.), 1× enhancer solution (EpiCentre Technologies, Austin Tex.), 1 unit Taq polymerase. The plate was sealed using a polypropylene plate sealer. The plate reaction was prepared and sealed at 4° C. and then transferred to a hot (95° C.) thermocycler (MWG, NC). The plate was subjected to a thermocycling procedure known as a touchdown procedure starting at 70° C. and ending at 60° C. over 30 cycles. At the end of 30 cycles, the plate was taken to 4° C.

The following mixture was combined and then 15 microliters of the mixture as obtained from either of the two above-described PCR procedures was added to each well in the microtiter plate:

250 μL 10× Thermopol buffer (NEB Biolabs, Beverly Mass.)

125 μL 10× N.BstNBI (NEB Biolabs, Beverly Mass.)

100 μL 25 mM dNTPs (NEB Biolabs, Beverly Mass.)

1000 μL 1 M trehalose (Sigma, St. Louis Mo.)

250 units N.BstNBI nicking enzyme (NEB Biolabs, Beverly Mass.)

50 units Vent exo- DNA polymerase (NEB Biolabs, Beverly Mass.)

1020 μL ultrapurewater

The plate was resealed at 4° C., and then the plate was heated to 60° C. for 1 hour.

A stainless-steel MALDI card was obtained from Micromass LTD (Manchester UK). The card was designed to accommodate 96-wells or “reading spots.” The top 4 rows (48 wells in all (A1-D12)) were treated with 0.1% w/v PDADMAC in 0.2× SSC, pH 7.6. Each spot received approximately ½ μL of the PDADMAC solution. The spots were allowed to dry, and then the card was washed with 10 mL of HPLC-grade water (Fisher). The card was dryed on top of a MJ thermocycler at 50° C. for 5 minutes.

Approximately 0.2 μL of the genotyping reaction was added to each spot. The solution was incubated for 5 minutes at 28° C. The solution was decanted by aspiration and the spots were washed with HPLC-grade water three times, and then twice with 10 mg/mL ammonium citrate. The card was then dried at 50° C. and the matrix was added to the spot. The matrix was composed of 25 mg/mL 3-hydroxypicolinic acid (Fluka, St. Louis, Mo.) in 50:50 mixture of acetonitrile (ACN)/water and 0.05% Trifluoroacetic acid (TFA). Approximately ½ μL was added per spot. The spot was dried 5 minutes at 3° C.

The MALDI conditions were as follows: Signal sensitivity=100.0; Function 1: polarity=LDI+; Source voltage (v)=15014; Pulse voltage=1750; MCP detector voltage=1950; Laser energy=20%; Matrix suppression (amu)=493.2; TLF delay (ns)=500.0; scans in function=10; cycle time (secs)=0.600; Ionization mode=LD+; retention window (minutes)=0.00-60.00; Data type=accuruate mass; Function type=Maldi TOF; mass range=1000-2000. Instrument calibration: MS1 static=none; MS1 scanning=none; MS1 scan speed=none; MS2 static=none; MS1 scanning=none; MS2 scan speed=none. Coefficients: MS1 static=none; MS2 static=none; function 1=0.000040788799*x2+1.001781806596*x+0.230258255211, root mass. Sample period (ns)=0.5; signal sensitivity=100.0.

Results:

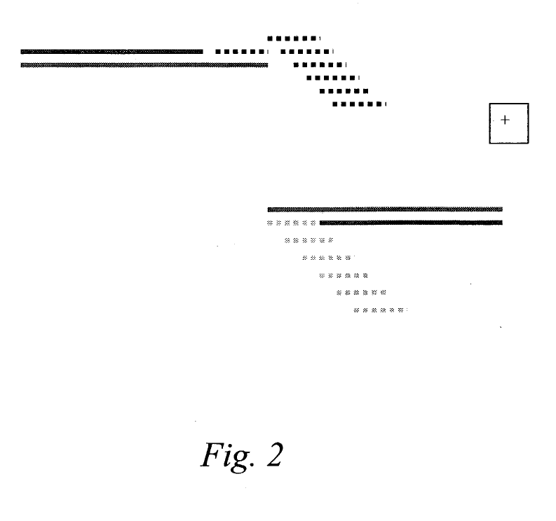

After nicking and amplification, the products illustrated in FIG. 2 are formed. In FIG. 2, the top solid fragment on the upper left duplex in the above figure has a molecular weight of 4006, the bottom solid fragment on the lower right duplex has a molecular weight of 4353, the bottom solid fragment on the upper left duplex has a molecular weight of 7014, the top solid fragment on the lower right duplex has a mass of 7837, the fragments indicated by dotted lines in the upper left duplex (the amplified fragments) have a mass of 3693, and the fragments indicated by the dotted lines in the lower right duplex hazel (the amplified fragments) have a mass of 7837.

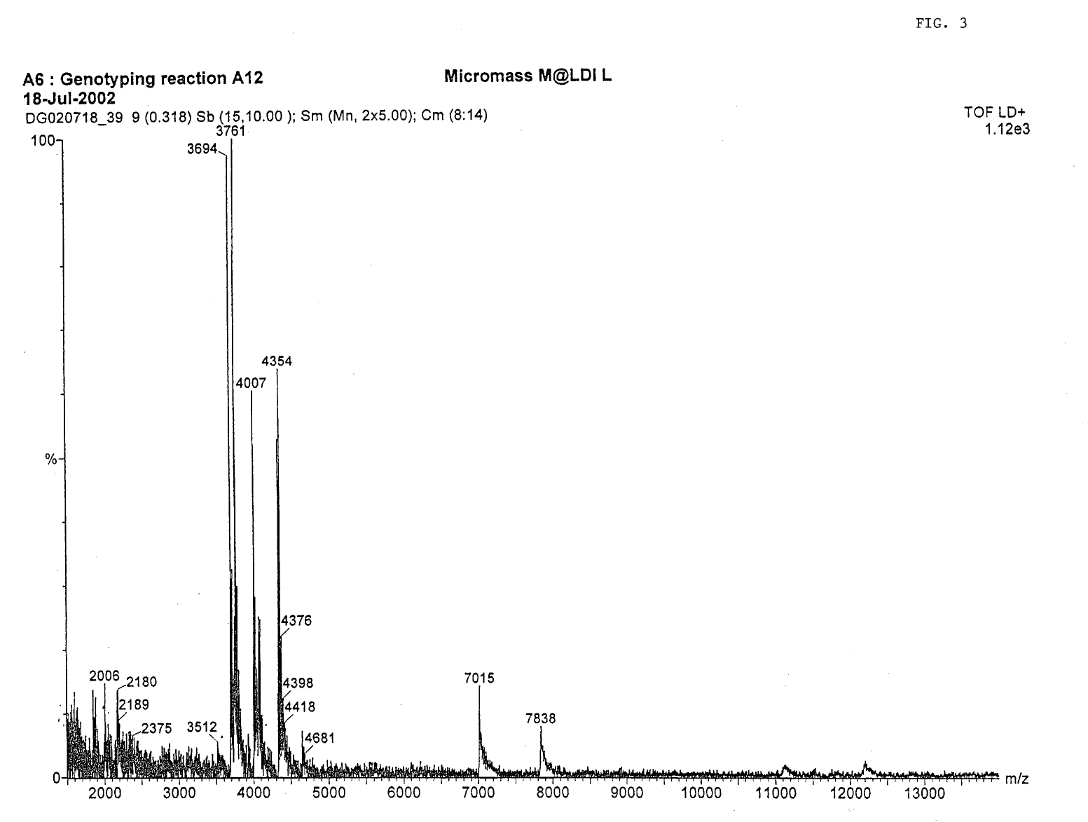

In FIG. 3, masses at 3691, 3761, 4007, 4354, 7015, and 7838 are clearly observed. The mass to charge ratios for the 12-mers (3691 and 3761) correspond to the SNP being the G allele (see electrospray results above).

From the foregoing it will be appreciated that, although specific embodiments of the invention have been described herein for purposes of illustration, various modifications may be made without deviating from the spirit and scope of the invention.

In this specification and in the appended claims, the singular forms “a”, “an,” and “the” include plural reference unless the context clearly dictates otherwise. Unless defined otherwise, all technical and scientific terms used herein have the same meanings as commonly understood to one of ordinary skill in the art to which this invention belongs.

Also, the following U.S. Patent Applications are incorporated herein by reference for all purposes: U.S. application Ser. No. 60/360,658 filed Feb. 28, 2002; and U.S. application Ser. No. 60/412,442, filed Sep. 20, 2002.

Accordingly, the invention is not limited except as by the appended claims.

1. A plate comprising a surface, the surface being at least partially coated with a coating comprising a cationic polyelectrolyte.

2. The plate of claim 1 wherein the cationic polyelectrolyte comprises a plurality of quaternary ammonium groups.

3. The plate of claim 2 wherein the cationic polyelectrolyte comprises the polymerization product of diallyldimethylammonium in association with a counterion.

4. The plate of claim 3 wherein the coating comprises poly(dimethyldiallyl ammonium) chloride (PDADMAC).

5. The plate of claim 1 having 10 to 1,000 wells.

6. The plate of claim 1 wherein the coating is in contact with polynucleotide.

7. The plate of claim 1 wherein the coating is in contact with an array of polynucleotides.

8. A dipstick comprising a surface, the surface being at least partially coated with a coating comprising a cationic polyelectrolyte.

9. The dipstick of claim 8 wherein the cationic polyelectrolyte comprises poly(dimethyidiallyl ammonium) chloride (PDADMAC).

10. A filter comprising a porous matrix, the matrix comprising a surface, the surface being at least partially coated with a coating comprising a cationic polyelectrolyte.

11. The filter of claim 10 wherein the cationic polyelectrolyte comprises poly(dimethyidiallyl ammonium) chloride (PDADMAC).

12. A method of performing an assay, comprising:
(a) providing a plate having wells with distinct indented surfaces, the surfaces coated with a cationic polyelectrolyte;
(b) performing a nucleotide extension assay in a well of (a);
(c) detecting the presence or absence of extension product.

13. The method of claim 12 wherein the cationic polyelectrolyte comprises poly(dimethyldiallyl ammonium) chloride (PDADMAC).

14. A method of isolating polynucleotides, comprising, in sequence:
(a) providing a solution comprising polynucleotides;
(b) inserting a substrate into the solution, the substrate at least partially coated with a cationic polyelectrolyte;
(c) allowing the polynucleotides to adhere to the cationic polyelectrolyte; and
(d) removing the substrate and at least some of the polynucleotides from the solution.

15. The method of claim 14 wherein the cationic polyelectrolyte comprises poly(dimethyldiallyl ammonium) chloride (PDADMAC).

16. A solid support comprising a surface, the surface at least partially coated with a cationic polyelectrolyte and an organic matrix.

17. The solid support of claim 16 wherein the support is in the form of a plate.

18. The solid support of claim 16, or the plate of claim 17, where the solid support or plate is at least partially formed from steel, glass fiber, glass, nylon 6/6, silicon, polyethylene, polypropylene, polyamide, polyvinylidenedifluoride, gold, silver, aluminum, copper, and plastic.

19. The solid support of claim 16 wherein the support is in the form of a multiwell plate.

20. The multiwell plate of claim 19 having 10 to 1,000 wells.

21. The solid support of claim 16 wherein the support is in the form of beads.

22. The beads of claim 21 wherein the bead material comprises silica, glass, a magnetic material, Sephadex, Sepharose, cellulose, stainless-steel or organic polymer.

23. The solid support of claim 16 in the form of an array of pins.

24. A method of performing an assay, comprising
(a) providing a solid support comprising a surface, the surface at least partially coated with a cationic polyelectrolyte;
(b) applying a polynucleotide solution to the coated surface;
(c) allowing the polynucleotide solution to interact with the polymer;
(d) washing the polymer/polynucleotide complex;
(e) applying an organic matrix solution; and
(f) measuring the polynucleotide by MALDI-MS.

25. The method of claim 24 wherein the cationic polyelectrolyte comprises diallydimethylammonium residues.

26. The method of claim 24 wherein the polynucleotide solution comprises a plurality of distinct polynucleotides.

27. The method of claim 24 wherein the wash consists of water washings and at least one washing with an ammonium salt solution.

28. A method of performing an assay, comprising
(a) providing a solid support comprising a surface, the surface at least partially coated with a cationic polyelectrolyte;
(b) applying a polynucleotide solution to the coated surface;
(c) allowing the polynucleotide solution to interact with the polymer;
(d) washing the polymer/polynucleotide complex; and
(e) measuring the polynucleotide by MALDI-MS.

29. The method of claim 28 wherein the cationic polyelectrolyte comprises diallydimethylammonium residues.

30. A method of performing an assay, comprising
(a) combining an analyte with a cationic polyelectrolyte to provide a mixture;
(b) placing the mixture on a substrate to provide a sample;
(c) analyzing the sample by MALDI-MS.

31. The method of claim 30 wherein the cationic polyelectrolyte comprises quaternary ammonium groups.

32. The method of claim 30 wherein the cationic polyelectrolyte is PDADMAC.

33. The method of claim 30 wherein matrix is present in the mixture.

34. The method of claim 30 wherein matrix is present in the sample.

35. A plate suitable for supporting a sample in a MALDI analysis, the plate comprising a coating of cationic polyelectrolyte.

36. The plate of claim 35 formed at least partially from stainless steel.

37. The plate of claim 35 wherein the cationic polyelectrolyte is PDADMAC.

38. The plate of claim 35 further comprising a matrix coating, where the matrix coating is able to transfer energy from an energy source to a sample on the plate.

39. The plate of claim 35 further comprising a biomolecule

40. The plate of claim 39 wherein the biomolecule is a polynucleotide.

41. The plate of claim 40 wherein the polynucleotide has 5-50 nucleotides.

42. The plate of claim 40 wherein the polynucleotide has 50-100 nucleotides.

43. A composition comprising a cationic polyelectrolyte and a MALDI matrix.

44. The composition of claim 43 further comprising a nucleic acid.

45. The composition of claim 43 further comprising protein.

46. The composition of claim 43 further comprising water.

47. The composition of claim 43 further comprising an organic solvent.

48. The composition of claim 47 wherein the organic solvent is acetonitrile.

49. A sample for MALDI analysis, the sample comprising a solid support comprising a surface, the surface at least partially covered with a coating, the coating comprising a cationic polyelctrolyte.

50. The sample of claim 49 wherein the coating further comprises a MALDI matrix.

51. The sample of claim 49 wherein the coating further comprises nucleic acid.

52. The sample of claim 49 wherein the solid support comprises a material selected from the group consisting of stainless steel, nickel, or platinum.

53. The sample of claim 49 wherein the solid support comprises an organic polymer.

54. A method of purifying nucleic acid, comprising
a. providing a mixture comprising nucleic acid and non-nucleic acid;
b. providing a solid support comprising a coating comprising cationic polyelectrolyte;
c. contacting the mixture with the cationic polyelectrolyte under conditions where nucleic acid adheres to the cationic polyelectrolyte, to provide adhered nucleic acid;
d. separating adhered nucleic acid from at least some of the non-nucleic acid; and
e. characterizing the adhered nucleic acid.

55. The method of claim 54 wherein the non-nucleic acid comprises protein, and adhered nucleic acid is separated from protein prior to the characterization of the adhered nucleic acid.

56. The method of claim 54 wherein adhered nucleic acid is separated from non-nucleic acid by washing the coating of adhered nucleic acid with water.

57. The method of claim 54 wherein the adhered nucleic acid is characterized by MALDI-MS.

58. The method of claim 54 wherein the adhered nucleic acid is contacted with matrix prior to being characterized by MALDI-MS.

59. A method for environmental analysis comprising
a) contacting an environment with a solid support comprising a surface, the surface being at least partially coated with a cationic polyelectrolyte, the cationic polyelectrolyte being contacted with the environment for a time sufficient for the cationic polyelectrolyte to absorb one or more organic compounds from the environment so as to provide a contaminated surface; and
b) analyzing the contaminated surface by MALDI-MS to provide an identification of the one or more organic compounds present in the environment.

60. The method of claim 59 wherein the environment is an atmosphere.

61. The method of claim 59 wherein the environment is a water supply.

62. The method of claims 59-61 wherein the cationic polyelectrolyte comprises quaternary amine groups.

63. The method of claim 62 wherein the cationic polyelectrolyte is PDADMAC.

64. A method for detecting the presence or absence of a biomolecule in an environment, comprising
a) exposing cationic polyelectrolyte to an environment, where the environment may or may not contain an organic biomolecule;
b) allowing the cationic polyelectrolyte to interact with the environment so as to absorb a biomolecule from the environment in the event a biomolecule is present in the environment, thereby providing a potentially contaminated cationic polyelectrolyte; and
c) analyzing the potentially contaminated cationic polyelectrolyte by MALDI-MS to determine whether an organic biomolecule was present in the environment.

65. The method of claim 64 wherein the organic biomolecule is selected from amino acid, saccharide and nucleotide.

66. The method of claim 64 wherein the organic biomolecule contains a plurality of residues selected from amino acid, saccharide and nucleotide.

67. The method of claim 64 wherein the organic biomolecule is selected from polypeptide, polysaccharide and polynucleotide.

68. The method of claim 64 wherein the organic biomolecule is indicative of a health hazard in the environment.

69. The method of claim 64 wherein the organic biomolecule is indicative of a pathogen.

70. The method of claim 64 wherein the cationic polyelectrolyte is part of a composite structure.

71. The method of claim 70 wherein the composite structure comprises a solid support and a coating of cationic polyelectrolyte on the solid support.

72. The method of claim 64 wherein the environment is an air supply.

73. The method of claim 64 wherein the environment is a water supply.

74. The method of claims 64-73 wherein the cationic polyelectrolyte comprises quaternary ammonium groups.

75. The method of claim 74 wherein the cationic poyelectrolyte is PDADMAC.

76. A kit for detecting the presence of an organic biomolecule in an air supply, comprising:
a) a solid support comprising a cationic polyelectrolyte; and
b) a means for securing the solid support in the air supply.

77. The kit of claim 76 wherein the cationic polyelectrolyte comprises quaternary ammonium groups.

78. The kit of claim 77 wherein the cationic polyelectrolyte is PDADMAC.

79. A kit for detecting the presence of an organic biomolecule in a water supply, comprising:
a) a solid support comprising a cationic polyelectrolyte; and
b) a means for securing the solid support in the water supply.

80. The kit of claim 79 wherein the cationic polyelectrolyte comprises quaternary ammonium groups.

81. The kit of claim 80 wherein the cationic polyelectrolyte is PDADMAC.
